SEQUENCE LISTING (13 sequences)

SEQ ID NO: 1 - DNA, 5423 bp, Homo sapiens
cccgggccaccgcctccgcccggctgcccgcccggactgtcgcggcccgcggtggcgacggcggccgctgcaaagtttccccggcggcggcggcccgggggcgcatcctcccgcaactgtcaagcgctggcggcggaaatgatgaggcgctggccattttccgagcccgggtttcctgcctgagccccgctcgagcgagccgcgagcgaggagccggcgggcgggagaggacgcgcccagggcgggggcccgcccgccccctcgggatttcgagggcccgggggcgcgcgacgccatgggccggccgggcccagagctcctgtctctcagcccggccgcaccacctgggtctccgccatgaacgggcctgccctgcagccctcctcgccctcttccgcgccctcagcctccccggcggcggccccgcggggctggagcgagttctgtgagttgcacgccgtagcggcggcccgggagctggcccgccagtactggctgttcgcccgggagcatccgcagcacgcgccgctgcgcgccgagctggtgtcgctgcagttcaccgacctcttccagcgctacttctgccgcgaggtgcgcgacggacgggcgccgggccgcgactaccgggacacaggccgtgggcccccagccaaggccgaggcgtccccggagccaggccccggccccgccgcccctggcctgcccaaggcccgcagctctgaggagctggccccgccgcggccgcccgggccctgctccttccagcactttcgccgcagcctccgccacatcttccgccgccgctcggccggggagctgccagcggcccacaccgctgccgcccccgggacccccggagaggctgctgagacccccgcccggcctggcctggccaagaagttcctgccctggagcctggcccgggagccgccacccgaggcgctgaaggaggcggtgctgcgctacagcctggccgacgaggcctccatggacagcggggcacgctggcagcgcgggaggctggcgctgcgccgggccccgggccccgatggccccgaccgcgtgctggagctcttcgacccacccaagagttcaaggcccaagctacaagcagcttgctccagcatccaggaggtccggtggtgcacacggcttgagatgcctgacaacctttacacctttgtgctgaaggtgaaggaccggacagacatcatctttgaggtgggagacgagcagcagctgaattcatggatggctgagctctcggagtgcacaggccgagggctggagagcacagaagcagagatgcatattccctcagccctagagcctagcacgtccagctccccaaggggcagcacagattcccttaaccaaggtgcttctcctggggggctgctggacccggcctgccagaagacggaccatttcctgtcctgctacccctggttccacggccccatctccagagtgaaagcagctcagctggttcagctgcagggccctgatgctcatggagtgttcctggtgcggcagagcgagacgcggcgtggggaatacgtgctcactttcaactttcaggggatagccaagcacctgcgcctgtcgctgacagagcggggccagtgccgtgtgcagcacctccactttccctcggtcgtggacatgctccaccacttccagcgctcgcccatcccactcgagtgcggcgccgcctgtgatgtccggctctccagctacgtggtagtcgtctcccaaccaccaggttcctgcaacacggtcctcttccctttctcccttcctcactgggattcagagtcccttcctcactggggttcagagttgggccttccccaccttagttcttctggctgtccccgggggctcagcccagagggtctcccagggcgatcctcaccccccgagcagatcttccacctggtgccttcgcccgaagaactggccaacagcctgcagcacctggagcatgagcctgtgaatcgagcccgggactcggactacgaaatggactcatcctcccggagccacctgcgggccatagacaatcagtacacacctctctgaccagtgaggaattccaggcctcaacagctgcccttgaggagcacaggcagaagtgtgaacttgtgaatgtaattgatctttccttccttccagagaaagatttaagggacactgttaactgctcgtgccagtttggaagtgacccttctattaggcctgttgaagggccctcctgtaggtttcatctatccacctggctttctccttattgtttacagatgtagttcttgttagaggatgccgctagctcctgcccggggtccctatgcccagtccccgttactcttagagaaaggagttggggtgagggccagagctggcagtggaaacttgttctctttttcactgacactgtcacagcggatgacagactttctacggggaggagggggggatcatcaggaagcccagaacactaacaagcggttctcccatctaccgtcagtccacatggcaggtctgctgtgtccacaccacagatgaccacatctaatcctgcttctactctcagctttaggacaaaagctctgtcagaggcacaagctgaaggtcaaaaatgatttaaaacattttacctcagactaatttctttaaaggattcaggttcaaaacttaaccactgcttatttcagtgcactgtttcaactaacacccatgctatttttgtagtcagaaacagctatgcaaaccctacctaatttacagtctgagccagcatgctggcttgtctactgcatcctcgggacagtcacctgccactgagtggccactgtccttcctaaatgtcaagaagtgaagtatgtcaccctttcagggaaattcaggcaattactgaaataggaaggtggcaagaacagttctatcctggtgccttacgaataaaaaactggattctggtttacagcagctttacagtgatagttaaattaactggggctaggggaaaagcaaccaaaaagggaaaaaggactcctaggccctttctattaaatccttcagcaacaaggctggcttggtgccctccaagcatctaatggcttattaaattatcccacaattgggttttaggctccttttttgacccaaaatggaagctgggaatctggtgccataactaatgagaaactcctttaataccccacaatcagtgttctgttctacctggctactgcttcactggattgaaaatctatctatctccttgcacacatgggcacacacaatctccaccatccagggaggtcctgaattcaaatctctatctatccaagtgatacaattcatagggggctggctcctcccagaacctgtctggaggctcagaaacgggggcagtgacagtggagtcagctgctcttgggtgccagcagagccattcagtacaacccccaggctcacagcagtggcttctaggaaactgggagtttagatcagctttacagatacatcgatcagaggctaaaatgaaacctcagcctaaaactcataggactgactgcctgggaggagggttaggtctgcttcttccacttatacttagtctctgtgctccaagaggtcaaatttttgcttctagaatttccttggggtctttcagagggtgggggaacaaacccctatgcacttttcttttttttttttgagatggagtttctcttgtcaaccgggctggagtgcagtggtgcaatcttggctcactgcaacctccaccttcctggttcaagcgattctgcctcgacctctcaagtagctgggattacaagcaccagccaccatgcctggctaattttgtatttttagtagagacagggtttcaccatgttggccaggctggtctcgaatgtctgacctcaggtgatccacccgccttggcctcccaaagtgctgggattacaggcgcgagccaccgcgcccagcctacaccacttttagtaccaacactcttgggtgatttcatggaccctaaagcagacctgacactgatccagatttgcagtccatttttaaggacacctgtctttatttcctcaaagtcaagcagctttctctggaaaatgaatgctaattagtgtgaaccaaaagagtaagtaagagtctgaagtttttttaaaggagaaagcttattatggaaagtcactggtcctcccctccgcacaggaaaggtacccagtagataatgaaccaaattaagttccctccctccagccagaagttaaacatctgggatatgacgtcttcatgccaggggcactcatttcttagcagcctctctacatacatctctcaggtggtgccaagaggcacaccaggtagagcaaacttagcagctctgactaacaggctgcaaagtgcaagttcagattctgtggcagagatttggagggcacccacgtccagactgcttcccgtccaagttaccaggacagctcaaaaacatgctgacagaaaactcccatggctctaggaaaaagtgacactaagccaacacctttctttatgtgggagcaaaatcagctgatgaaggggtgggcaccattgtggggcaggcaccccactggctgcagctagcccaccataggcacagcacatcccaccactctccttccagtcctgaccaggccccagccggcaacttctaccgagagccatggctcaacaccaaactggacagtagacatcatgatccctccagttagctctaattacagaccccaccagtacagcttgacagctcccggcaccatcccttccttcatctgacttattgaacttttacaaactaacagtcaccagcaccaaagaattaagtcaactaacctgccttgaattttaaaccagcaatccatatggctttatctggtataaatcttctgcctttgatcatttctggaccgtaggaaaaaggaatagcaatcattaaaatcttgggccagagaacactatttttacataacagtttcttaacctaaagtcaaggccttggactcttccctgagggttgcctgaaattccttcatgctttctattcaggactaattcccttactgcaaatgtgttagctctaacatctcccacaagctaaaggaacttgcaagtatattaacaaggacacatctgacatcctgtgtttggttaaaatatacagcacattgtgataacataaagtggatccatcttgtatcattataggcaaaaggtatttggcaaatttttatgtatggttttatgtactgtacaagtaacttattcttgaataatgcaaattttgctataatgtacaaattgctatatgtgaattaaaaagtttccaaaatcttgaaaaaaaaaaaaaaaaaaaa

SEQ ID NO: 2 - DNA, 5423 bp, Homo sapiens
ttttttttttttttttttttcaagattttggaaactttttaattcacatatagcaatttgtacattatagcaaaatttgcattattcaagaataagttacttgtacagtacataaaaccatacataaaaatttgccaaataccttttgcctataatgatacaagatggatccactttatgttatcacaatgtgctgtatattttaaccaaacacaggatgtcagatgtgtccttgttaatatacttgcaagttcctttagcttgtgggagatgttagagctaacacatttgcagtaagggaattagtcctgaatagaaagcatgaaggaatttcaggcaaccctcagggaagagtccaaggccttgactttaggttaagaaactgttatgtaaaaatagtgttctctggcccaagattttaatgattgctattcctttttcctacggtccagaaatgatcaaaggcagaagatttataccagataaagccatatggattgctggtttaaaattcaaggcaggttagttgacttaattctttggtgctggtgactgttagtttgtaaaagttcaataagtcagatgaaggaagggatggtgccgggagctgtcaagctgtactggtggggtctgtaattagagctaactggagggatcatgatgtctactgtccagtttggtgttgagccatggctctcggtagaagttgccggctggggcctggtcaggactggaaggagagtggtgggatgtgctgtgcctatggtgggctagctgcagccagtggggtgcctgccccacaatggtgcccaccccttcatcagctgattttgctcccacataaagaaaggtgttggcttagtgtcactttttcctagagccatgggagttttctgtcagcatgtttttgagctgtcctggtaacttggacgggaagcagtctggacgtgggtgccctccaaatctctgccacagaatctgaacttgcactttgcagcctgttagtcagagctgctaagtttgctctacctggtgtgcctcttggcaccacctgagagatgtatgtagagaggctgctaagaaatgagtgcccctggcatgaagacgtcatatcccagatgtttaacttctggctggagggagggaacttaatttggttcattatctactgggtacctttcctgtgcggaggggaggaccagtgactttccataataagctttctcctttaaaaaaacttcagactcttacttactcttttggttcacactaattagcattcattttccagagaaagctgcttgactttgaggaaataaagacaggtgtccttaaaaatggactgcaaatctggatcagtgtcaggtctgctttagggtccatgaaatcacccaagagtgttggtactaaaagtggtgtaggctgggcgcggtggctcgcgcctgtaatcccagcactttgggaggccaaggcgggtggatcacctgaggtcagacattcgagaccagcctggccaacatggtgaaaccctgtctctactaaaaatacaaaattagccaggcatggtggctggtgcttgtaatcccagctacttgagaggtcgaggcagaatcgcttgaaccaggaaggtggaggttgcagtgagccaagattgcaccactgcactccagcccggttgacaagagaaactccatctcaaaaaaaaaaaagaaaagtgcataggggtttgttcccccaccctctgaaagaccccaaggaaattctagaagcaaaaatttgacctcttggagcacagagactaagtataagtggaagaagcagacctaaccctcctcccaggcagtcagtcctatgagttttaggctgaggtttcattttagcctctgatcgatgtatctgtaaagctgatctaaactcccagtttcctagaagccactgctgtgagcctgggggttgtactgaatggctctgctggcacccaagagcagctgactccactgtcactgcccccgtttctgagcctccagacaggttctgggaggagccagccccctatgaattgtatcacttggatagatagagatttgaattcaggacctccctggatggtggagattgtgtgtgcccatgtgtgcaaggagatagatagattttcaatccagtgaagcagtagccaggtagaacagaacactgattgtggggtattaaaggagtttctcattagttatggcaccagattcccagcttccattttgggtcaaaaaaggagcctaaaacccaattgtgggataatttaataagccattagatgcttggagggcaccaagccagccttgttgctgaaggatttaatagaaagggcctaggagtcctttttccctttttggttgcttttcccctagccccagttaatttaactatcactgtaaagctgctgtaaaccagaatccagttttttattcgtaaggcaccaggatagaactgttcttgccaccttcctatttcagtaattgcctgaatttccctgaaagggtgacatacttcacttcttgacatttaggaaggacagtggccactcagtggcaggtgactgtcccgaggatgcagtagacaagccagcatgctggctcagactgtaaattaggtagggtttgcatagctgtttctgactacaaaaatagcatgggtgttagttgaaacagtgcactgaaataagcagtggttaagttttgaacctgaatcctttaaagaaattagtctgaggtaaaatgttttaaatcatttttgaccttcagcttgtgcctctgacagagcttttgtcctaaagctgagagtagaagcaggattagatgtggtcatctgtggtgtggacacagcagacctgccatgtggactgacggtagatgggagaaccgcttgttagtgttctgggcttcctgatgatccccccctcctccccgtagaaagtctgtcatccgctgtgacagtgtcagtgaaaaagagaacaagtttccactgccagctctggccctcaccccaactcctttctctaagagtaacggggactgggcatagggaccccgggcaggagctagcggcatcctctaacaagaactacatctgtaaacaataaggagaaagccaggtggatagatgaaacctacaggagggcccttcaacaggcctaatagaagggtcacttccaaactggcacgagcagttaacagtgtcccttaaatctttctctggaaggaaggaaagatcaattacattcacaagttcacacttctgcctgtgctcctcaagggcagctgttgaggcctggaattcctcactggtcagagaggtgtgtactgattgtctatggcccgcaggtggctccgggaggatgagtccatttcgtagtccgagtcccgggctcgattcacaggctcatgctccaggtgctgcaggctgttggccagttcttcgggcgaaggcaccaggtggaagatctgctcggggggtgaggatcgccctgggagaccctctgggctgagcccccggggacagccagaagaactaaggtggggaaggcccaactctgaaccccagtgaggaagggactctgaatcccagtgaggaagggagaaagggaagaggaccgtgttgcaggaacctggtggttgggagacgactaccacgtagctggagagccggacatcacaggcggcgccgcactcgagtgggatgggcgagcgctggaagtggtggagcatgtccacgaccgagggaaagtggaggtgctgcacacggcactggccccgctctgtcagcgacaggcgcaggtgcttggctatcccctgaaagttgaaagtgagcacgtattccccacgccgcgtctcgctctgccgcaccaggaacactccatgagcatcagggccctgcagctgaaccagctgagctgctttcactctggagatggggccgtggaaccaggggtagcaggacaggaaatggtccgtcttctggcaggccgggtccagcagccccccaggagaagcaccttggttaagggaatctgtgctgccccttggggagctggacgtgctaggctctagggctgagggaatatgcatctctgcttctgtgctctccagccctcggcctgtgcactccgagagctcagccatccatgaattcagctgctgctcgtctcccacctcaaagatgatgtctgtccggtccttcaccttcagcacaaaggtgtaaaggttgtcaggcatctcaagccgtgtgcaccaccggacctcctggatgctggagcaagctgcttgtagcttgggccttgaactcttgggtgggtcgaagagctccagcacgcggtcggggccatcggggcccggggcccggcgcagcgccagcctcccgcgctgccagcgtgccccgctgtccatggaggcctcgtcggccaggctgtagcgcagcaccgcctccttcagcgcctcgggtggcggctcccgggccaggctccagggcaggaacttcttggccaggccaggccgggcgggggtctcagcagcctctccgggggtcccgggggcggcagcggtgtgggccgctggcagctccccggccgagcggcggcggaagatgtggcggaggctgcggcgaaagtgctggaaggagcagggcccgggcggccgcggcggggccagctcctcagagctgcgggccttgggcaggccaggggcggcggggccggggcctggctccggggacgcctcggccttggctgggggcccacggcctgtgtcccggtagtcgcggcccggcgcccgtccgtcgcgcacctcgcggcagaagtagcgctggaagaggtcggtgaactgcagcgacaccagctcggcgcgcagcggcgcgtgctgcggatgctcccgggcgaacagccagtactggcgggccagctcccgggccgccgctacggcgtgcaactcacagaactcgctccagccccgcggggccgccgccggggaggctgagggcgcggaagagggcgaggagggctgcagggcaggcccgttcatggcggagacccaggtggtgcggccgggctgagagacaggagctctgggcccggccggcccatggcgtcgcgcgcccccgggccctcgaaatcccgagggggcgggcgggcccccgccctgggcgcgtcctctcccgcccgccggctcctcgctcgcggctcgctcgagcggggctcaggcaggaaacccgggctcggaaaatggccagcgcctcatcatttccgccgccagcgcttgacagttgcgggaggatgcgcccccgggccgccgccgccggggaaactttgcagcggccgccgtcgccaccgcgggccgcgacagtccgggcgggcagccgggcggaggcggtggcccggg

SEQ ID NO: 3 - protein, 575 aa, Homo sapiens
MNGPALQPSSPSSAPSASPAAAPRGWSEFCELHAVAAARELARQYWLFAREHPQHAPLRAELVSLQFTDLFQRYFCREVRDGRAPGRDYRDTGRGPPAKAEASPEPGPGPAAPGLPKARSSEELAPPRPPGPCSFQHFRRSLRHIFRRRSAGELPAAHTAAAPGTPGEAAETPARPGLAKKFLPWSLAREPPPEALKEAVLRYSLADEASMDSGARWQRGRLALRRAPGPDGPDRVLELFDPPKSSRPKLQAACSSIQEVRWCTRLEMPDNLYTFVLKVKDRTDIIFEVGDEQQLNSWMAELSECTGRGLESTEAEMHIPSALEPSTSSSPRGSTDSLNQGASPGGLLDPACQKTDHFLSCYPWFHGPISRVKAAQLVQLQGPDAHGVFLVRQSETRRGEYVLTFNFQGIAKHLRLSLTERGQCRVQHLHFPSVVDMLHHFQRSPIPLECGAACDVRLSSYVVVVSQPPGSCNTVLFPFSLPHWDSESLPHWGSELGLPHLSSSGCPRGLSPEGLPGRSSPPEQIFHLVPSPEELANSLQHLEHEPVNRARDSDYEMDSSSRSHLRAIDNQYTPL

SEQ ID NO: 4 - DNA, 1888 bp, Mus musculus
ggcacgagatcccattacagatggttgtgagccaccatgtggttgctgggatttgaactcaggacctctggaagagcagtcagtactcttaaccacagagccgttggagtggctggcatgtgacctgaaactggagtgccagactgccttgccctgttgccaagcccggccgccccgcctcgctctccaccatgaacgagcccaccgtgcagccgtcccgcacatcctccgcacccgcctcgccggcatccccacgcggctggagcgacttctgcgagcagcacgcagcagcggcggcccgggagctggcccgccagtactggttgtttgcgcgcgcgcacccacagccgccgcgcgcggacctggtgtcgctgcagttcgcggagctcttccagcgccacttctgccgggaggtgcgcgagagcctcgcaggaccgccgggtcacgactaccgcgccactgctccgccccgccccgcgctgcccaaggcacgcagctccgaggacctgggcccgcggcccgcctgtgccctgcagcacctgcgccgcggcctgcgccagctcttccgccgccgctcggcaggggagctgcccggggctaccagtgacaccaatgacatcgacaccaccgcagccagcaggccgggcccggcccgcaagttgctaccctggggcctgcgagagccgcccactgaggcgctcaaggaggtcgtattgcgctatagcctggcggacgaggcagcaatggacagcggcgcacgctggcagcggggtcgcctggtgcttcggtctccaggtccgggccacagccactttctgcagctcttcgatccgcccaagagctcaaagcccaagctccaagaggcctgttccagcatccgggaggtccgaccatgtacacgcctggagatgcctgacaacctctacacctttgtgttgaaggtgcaggaccagacagacatcatctttgaggtgggagatgaacagcagctgaactcatggctggcagagctcagggcaagcacaggccttgggctggagcacccggacaccgagttacctctttccttagcggcagagcctggcccagctagatccccaaggggaagcactgactccctggaccaaggtgcttcacctggggtgttgctggacccagcctgccagaaaacagatcacttcctatcctgctacccctggttccacggccccatctccagggtgagggctgcacagctggtccagctccagggccctgatgcccacggcgtgttcctggtgcggcagagtgagtcccggagaggagagtatgtactcacattcaacttacagggcagagccaagcacttacgcctggtgctcacagagcgtggacagtgccgggtgcaacacctgcacttcccctcggtggtagatatgctccgccacttccagcgttctcctatcccactggaatgtggagcagcttgtgacgtccgactctctggctatgtggtagtcctctctcaggcaccaggttcctccaacaccgtcctcttccctttttcccttcctcactgggattcggagctgggtcatccccacctcagctctgttggctgtccccccagccatggtgcagaggctctccctggccaagtgacaccacctgagcagatcttccacctggtgccttctcctgaggaactggccaacagtctgcggcagctggagctcgagtctgtgagcagtgcccgggactcggactatgacatggactcctcttcacggggccaccttcgggccattgacaaccagtacacccctctctcacagctgtgcagagaggcagacgtgtgaatggaaccattttcctcccctccagagaactataggctgctgtcagcctc

SEQ ID NO: 5 - DNA, 1888 bp, Mus musculus
gaggctgacagcagcctatagttctctggaggggaggaaaatggttccattcacacgtctgcctctctgcacagctgtgagagaggggtgtactggttgtcaatggcccgaaggtggccccgtgaagaggagtccatgtcatagtccgagtcccgggcactgctcacagactcgagctccagctgccgcagactgttggccagttcctcaggagaaggcaccaggtggaagatctgctcaggtggtgtcacttggccagggagagcctctgcaccatggctggggggacagccaacagagctgaggtggggatgacccagctccgaatcccagtgaggaagggaaaaagggaagaggacggtgttggaggaacctggtgcctgagagaggactaccacatagccagagagtcggacgtcacaagctgctccacattccagtgggataggagaacgctggaagtggcggagcatatctaccaccgaggggaagtgcaggtgttgcacccggcactgtccacgctctgtgagcaccaggcgtaagtgcttggctctgccctgtaagttgaatgtgagtacatactctcctctccgggactcactctgccgcaccaggaacacgccgtgggcatcagggccctggagctggaccagctgtgcagccctcaccctggagatggggccgtggaaccaggggtagcaggataggaagtgatctgttttctggcaggctgggtccagcaacaccccaggtgaagcaccttggtccagggagtcagtgcttccccttggggatctagctgggccaggctctgccgctaaggaaagaggtaactcggtgtccgggtgctccagcccaaggcctgtgcttgccctgagctctgccagccatgagttcagctgctgttcatctcccacctcaaagatgatgtctgtctggtcctgcaccttcaacacaaaggtgtagaggttgtcaggcatctccaggcgtgtacatggtcggacctcccggatgctggaacaggcctcttggagcttgggctttgagctcttgggcggatcgaagagctgcagaaagtggctgtggcccggacctggagaccgaagcaccaggcgaccccgctgccagcgtgcgccgctgtccattgctgcctcgtccgccaggctatagcgcaatacgacctccttgagcgcctcagtgggcggctctcgcaggccccagggtagcaacttgcgggccgggcccggcctgctggctgcggtggtgtcgatgtcattggtgtcactggtagccccgggcagctcccctgccgagcggcggcggaagagctggcgcaggccgcggcgcaggtgctgcagggcacaggcgggccgcgggcccaggtcctcggagctgcgtgccttgggcagcgcggggcggggcggagcagtggcgcggtagtcgtgacccggcggtcctgcgaggctctcgcgcacctcccggcagaagtggcgctggaagagctccgcgaactgcagcgacaccaggtccgcgcgcggcggctgtgggtgcgcgcgcgcaaacaaccagtactggcgggccagctcccgggccgccgctgctgcgtgctgctcgcagaagtcgctccagccgcgtggggatgccggcgaggcgggtgcggaggatgtgcgggacggctgcacggtgggctcgttcatggtggagagcgaggcggggcggccgggcttggcaacagggcaaggcagtctggcactccagtttcaggtcacatgccagccactccaacggctctgtggttaagagtactgactgctcttccagaggtcctgagttcaaatcccagcaaccacatggtggctcacaaccatctgtaatgggatctcgtgcc

SEQ ID NO: 6 - protein, 548 aa, Mus musculus
MNEPTVQPSRTSSAPASPASPRGWSDFCEQHAAAAARELARQYWLFARAHPQPPRADLVSLQFAELFQRHFCREVRESLAGPPGHDYRATAPPRPALPKARSSEDLGPRPACALQHLRRGLRQLFRRRSAGELPGATSDTNDIDTTAASRPGPARKLLPWGLREPPTEALKEVVLRYSLADEAAMDSGARWQRGRLVLRSPGPGHSHFLQLFDPPKSSKPKLQEACSSIREVRPCTRLEMPDNLYTFVLKVQDQTDIIFEVGDEQQLNSWLAELRASTGLGLEHPDTELPLSLAAEPGPARSPRGSTDSLDQGASPGVLLDPACQKTDHFLSCYPWFHGPISRVRAAQLVQLQGPDAHGVFLVRQSESRRGEYVLTFNLQGRAKHLRLVLTERGQCRVQHLHFPSVVDMLRHFQRSPIPLECGAACDVRLSGYVVVLSQAPGSSNTVLFPFSLPHWDSELGHPHLSSVGCPPSHGAEALPGQVTPPEQIFHLVPSPEELANSLRQLELESVSSARDSDYDMDSSSRGHLRAIDNQYTPLSQLCREADV

SEQ ID NO: 7 - DNA, 29 bp, Mus musculus
aaggatccatgcctgacaacctctacacc

SEQ ID NO: 8 - DNA, 29 bp, Mus musculus
atggatcctaggggtagcaggataggaag

SEQ ID NO: 9 - DNA, 27 bp, Mus musculus
ttggatccgtcctctctcaggcaccag

SEQ ID NO: 10 - DNA, 26 bp, Mus musculus
taggatccattcacacgtctgcctct

SEQ ID NO: 11 - DNA, 3680 bp, Rattus norvegicus
tcctagagctcaaagcccaagctccaagaggcctgctccagcatccaggaggtccgaccctgcacacgcctggagatgccggacaacctctacacctttgtgttgaaggtgagcgccagggctgtggctccttggggagacactgctgtctcctcaggtgcagagctatatgcctcctttctgcaggtgcagggccagacagacatcatctttgaggtgggagatgaacagcagctgaactcatggctggcagaactcagggcaagcacaggcctagggtgaggacctgcccatccctggttccctttgataacccctgctggcccccttctcaccagcctcttcccctacaggttggagcacctggacacagagttacctctttccttagtggcagagcctggcccagctatatccccaaggggaagcacggactccctggaccaaggtatgtgccagatccttggtctctgggtgtccttggacatgtggcccctctgcgggtcaccaccaccatctttctgccaggtgcttcacctggggtgatgttggacccagcctgccagaaaacagatcacttcctgtcctgctacccctggttccacggccccatctccagggtgagggctgcacagctggttcagctgcagggccctgatgcccacggtgtgttcctggtgcggcagagtgagtcccggagaggggaatatgtactcacattcaacttacaggggagagccaagcacctccgcctggtgctcacagagcgtggacagtgccgggtgcaacacctgcacttcccctcggtggtggatatgctccgccacttccagcgctcccccatcccacttgaatgtggagcagcctgtgatgtccgactctctggctatgtggtagtcgtctctcaggcaccaggtaggaagcccaaagttgtgtcaaggggtggtgtcacagcaagtacagcacgtacaaaacttccttcctggttacgccttcttcatggagtcatccatgttatctctccaggttcctccaacaccgttctcttccccttttcccttcctcactgggactcagagctgggccatccccacctcagctctgctggctgtccccctggccatggtgcagaggctctccgtggccaagtgacacctcctgagcagatcttccacctggtgccttctcctgaggaactggccaacagtctgcggcagctggagcttgagtctgtgagcagtgcccgggactcagactatgaaatggactcctcctcacggagccaccttcgggccattgacaaccagtacacccctctctcacagctgtgcagagaggcaaacttgtgaatgtaaccattttccttccctccagagaactacaggctgctgctgtcagccttccccagctcagatgtgcctgcttccaccacacacaaggctgtcacaggcttctgtcagccacagctagcctggcttcttcccactgtctgtagatgtagttcttgtgcacaggtgccactagctggcaccaaggcctcgttcccaacccagaggaggcggggctgcaggcccagagctggcagtggaaactgacagagctgatgacagacttcttaccaggtagggtcatcgggaagcacaaaacactaacagtccctggattctcacatggtgtccttccctgtagcttcattctgtggcaagtctaagtcccatgtcacaggcgtaggacacttcataatcctgcttctactcttagctttggacagaagctctgccagaggggcaagcttaaagatcaaaaatgatttaaacattttacctcagagtaattatttttaaagggttcaggtttccacttgatattatagcttacttcactgcactgccctgctaccccctgccactgtaaagttggggggagctgagtccccctgctttgcttaccttgagccaggctagcatgctagtgtccttgtctgctcaccctcaggacagttgcctgctacggggtagacactgtcctccctaatgtagagaatgaagtgtgtcaccctctcagggaaatccaggcagcttgcagagagaggagggtggcaggggacattccaccccagtgccttatgcattaaaaacaaaatcaggactgtggtttacagcagcgttagaggatgagatgaatgggtgggggaagacaaacacagatggaaggcctctaggcttctttagtgaacccacattttactccccccaaccccctcccagcatctaggggcttagtgagtttggcccatgctatgaagaaactcctgtaaccgctcctaggtgttcttatccatacaatctttccatctagacagagccaaaaactgtcatctttcctaagggatgcagttcacagggactggagcaggctgttcatggcacaaacagtgcttacagcagtggctttgggaaaccacaacttaattccttgaacattgagaggcagccatgctgctgccctgctgcagacagcagtgctgactccccctctgctcaccttgcactcgggaagcctgcgtgggctttggtgatggaagcagctgcacctcagcttcgacacccaccacaggtgtggacctgtgctcatcttaggacagcgccttccttcactgcacatcaggtatgaaccagagacgtgagcagtaacagccagtgggccaggccggctctaatccacacaggatgcgtggacggctgtgccctgcacctggccaggagctaaacccacttcaaacttggggcactcatttctcctgggccttcaaagagagacttgctaggtgctgtaagccagcaccttggccccttagtgctgggattcactcactgtggacagctttgtttcaaaaaatgtccaagatatcagaaaacaaggtaaaaaagtctcatgactctgtgacagagtgacaacactaagccagcaccttttctttaagctaaagtaggtcagttggctgacggggacagacgggtaccgggtatgagaactcctgggccagataccctgtgtcaaagccaggccccactcccaccctcagcagtgtcttctggccacacaaggcaggatgggtcagtggccactagagacagagcagctcaacagtcctgaacccctgctcccttctcccgagtactcaactcagactaacaagcaccagcaccaaagagttaggtcaactaacctgccttgtctttaccagcatccacatggctttatctgtataaatcttctgcctttgatcatttctggatggtgtaggaaaaaaggaatagtgaccattacagtcttgggccagacaacactatttttacctaagtctcttaacctaaattacgtcgcctcttcctgaggctcagtctcttgtttaaatgttaccttggtgcaaataaaagtgataaactgtcccccactgcctcctgacgagacacatctgtctaatggccagcgtgtggctggaataagcagcattgtgaccacaggatcaggggacttcatcttctatcattagagcagaaggtatttggcaaatctttgttttatgtactgtacgagtaacttattcttgaataatgcaaattttcctacaatgtacaaattgctgtatgtgaattaaaaaggttttcagaattaca

SEQ ID NO: 12 - DNA, 3680 bp, Rattus norvegicus
tgtaattctgaaaacctttttaattcacatacagcaatttgtacattgtaggaaaatttgcattattcaagaataagttactcgtacagtacataaaacaaagatttgccaaataccttctgctctaatgatagaagatgaagtcccctgatcctgtggtcacaatgctgcttattccagccacacgctggccattagacagatgtgtctcgtcaggaggcagtgggggacagtttatcacttttatttgcaccaaggtaacatttaaacaagagactgagcctcaggaagaggcgacgtaatttaggttaagagacttaggtaaaaatagtgttgtctggcccaagactgtaatggtcactattccttttttcctacaccatccagaaatgatcaaaggcagaagatttatacagataaagccatgtggatgctggtaaagacaaggcaggttagttgacctaactctttggtgctggtgcttgttagtctgagttgagtactcgggagaagggagcaggggttcaggactgttgagctgctctgtctctagtggccactgacccatcctgccttgtgtggccagaagacactgctgagggtgggagtggggcctggctttgacacagggtatctggcccaggagttctcatacccggtacccgtctgtccccgtcagccaactgacctactttagcttaaagaaaaggtgctggcttagtgttgtcactctgtcacagagtcatgagacttttttaccttgttttctgatatcttggacattttttgaaacaaagctgtccacagtgagtgaatcccagcactaaggggccaaggtgctggcttacagcacctagcaagtctctctttgaaggcccaggagaaatgagtgccccaagtttgaagtgggtttagctcctggccaggtgcagggcacagccgtccacgcatcctgtgtggattagagccggcctggcccactggctgttactgctcacgtctctggttcatacctgatgtgcagtgaaggaaggcgctgtcctaagatgagcacaggtccacacctgtggtgggtgtcgaagctgaggtgcagctgcttccatcaccaaagcccacgcaggcttcccgagtgcaaggtgagcagagggggagtcagcactgctgtctgcagcagggcagcagcatggctgcctctcaatgttcaaggaattaagttgtggtttcccaaagccactgctgtaagcactgtttgtgccatgaacagcctgctccagtccctgtgaactgcatcccttaggaaagatgacagtttttggctctgtctagatggaaagattgtatggataagaacacctaggagcggttacaggagtttcttcatagcatgggccaaactcactaagcccctagatgctgggagggggttggggggagtaaaatgtgggttcactaaagaagcctagaggccttccatctgtgtttgtcttcccccacccattcatctcatcctctaacgctgctgtaaaccacagtcctgattttgtttttaatgcataaggcactggggtggaatgtcccctgccaccctcctctctctgcaagctgcctggatttccctgagagggtgacacacttcattctctacattagggaggacagtgtctaccccgtagcaggcaactgtcctgagggtgagcagacaaggacactagcatgctagcctggctcaaggtaagcaaagcagggggactcagctccccccaactttacagtggcagggggtagcagggcagtgcagtgaagtaagctataatatcaagtggaaacctgaaccctttaaaaataattactctgaggtaaaatgtttaaatcatttttgatctttaagcttgcccctctggcagagcttctgtccaaagctaagagtagaagcaggattatgaagtgtcctacgcctgtgacatgggacttagacttgccacagaatgaagctacagggaaggacaccatgtgagaatccagggactgttagtgttttgtgcttcccgatgaccctacctggtaagaagtctgtcatcagctctgtcagtttccactgccagctctgggcctgcagccccgcctcctctgggttgggaacgaggccttggtgccagctagtggcacctgtgcacaagaactacatctacagacagtgggaagaagccaggctagctgtggctgacagaagcctgtgacagccttgtgtgtggtggaagcaggcacatctgagctggggaaggctgacagcagcagcctgtagttctctggagggaaggaaaatggttacattcacaagtttgcctctctgcacagctgtgagagaggggtgtactggttgtcaatggcccgaaggtggctccgtgaggaggagtccatttcatagtctgagtcccgggcactgctcacagactcaagctccagctgccgcagactgttggccagttcctcaggagaaggcaccaggtggaagatctgctcaggaggtgtcacttggccacggagagcctctgcaccatggccagggggacagccagcagagctgaggtggggatggcccagctctgagtcccagtgaggaagggaaaaggggaagagaacggtgttggaggaacctggagagataacatggatgactccatgaagaaggcgtaaccaggaaggaagttttgtacgtgctgtacttgctgtgacaccaccccttgacacaactttgggcttcctacctggtgcctgagagacgactaccacatagccagagagtcggacatcacaggctgctccacattcaagtgggatgggggagcgctggaagtggcggagcatatccaccaccgaggggaagtgcaggtgttgcacccggcactgtccacgctctgtgagcaccaggcggaggtgcttggctctcccctgtaagttgaatgtgagtacatattcccctctccgggactcactctgccgcaccaggaacacaccgtgggcatcagggccctgcagctgaaccagctgtgcagccctcaccctggagatggggccgtggaaccaggggtagcaggacaggaagtgatctgttttctggcaggctgggtccaacatcaccccaggtgaagcacctggcagaaagatggtggtggtgacccgcagaggggccacatgtccaaggacacccagagaccaaggatctggcacataccttggtccagggagtccgtgcttccccttggggatatagctgggccaggctctgccactaaggaaagaggtaactctgtgtccaggtgctccaacctgtaggggaagaggctggtgagaagggggccagcaggggttatcaaagggaaccagggatgggcaggtcctcaccctaggcctgtgcttgccctgagttctgccagccatgagttcagctgctgttcatctcccacctcaaagatgatgtctgtctggccctgcacctgcagaaaggaggcatatagctctgcacctgaggagacagcagtgtctccccaaggagccacagccctggcgctcaccttcaacacaaaggtgtagaggttgtccggcatctccaggcgtgtgcagggtcggacctcctggatgctggagcaggcctcttggagcttgggctttgagctctagga

SEQ ID NO: 13 - protein, 368 aa, Rattus norvegicus
MAGRTQGKHRPRVRTCPSLVPFDNPCWPPSHQPLPLQVGAPGHRVTSFLSGRAWPSYIPKGKHGLPGPRYVPDPWSLGVLGHVAPLRVTTTIFLPGASPGVMLDPACQKTDHFLSCYPWFHGPISRVRAAQLVQLQGPDAHGVFLVRQSESRRGEYVLTFNLQGRAKHLRLVLTERGQCRVQHLHFPSVVDMLRHFQRSPIPLECGAACDVRLSGYVVVVSQAPGRKPKVVSRGGVTASTARTKLPSWLRLLHGVIHVISPGSSNTVLFPFSLPHWDSELGHPHLSSAGCPPGHGAEALRGQVTPPEQIFHLVPSPEELANSLRQLELESVSSARDSDYEMDSSSRSHLRAIDNQYTPLSQLCREANL

(19) United States
(12) Patent Application Publication
(10) Pub. No.: US 2010/0130423 A1
(43) Pub. Date: May 27, 2010

(54) TREATMENT OF MYELOPROLIFERATIVE DISORDERS WITH ADAPTOR PROTEIN LNK

(75) Inventors: H. Phillip Koeffler, Los Angeles, CA (US); Sigal Gery, Beverly Hills, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 12/598,093

(22) PCT Filed: Apr. 30, 2008
(86) PCT No.: PCT/US08/62111

§ 371 Date: Oct. 29, 2009

Related U.S. Application Data
(60) Provisional application No. 60/915,069, filed on Apr. 30, 2007.
(60) Provisional application No. 60/915,341, filed on May 1, 2007.

(51) Int. Cl. A61K 38/17; C12N 5/02

(52) U.S. Cl. 514/12; 435/375

(57) ABSTRACT

Janus kinase 2 (JAK2) associates with cytokine receptors and is essential for signal transduction in hematopoietic cells. The JAK2 mutation, JAK2 V617F, prevalent in myeloproliferative disorders, confers cytokine-independent proliferation and constitutive activation of downstream signaling pathways, when co-expressed with homodimeric type I cytokine receptors. The adaptor protein LnK is a negative regulator of hematopoietic cytokine receptors, including EPOR and MPL. LnK attenuates wild type JAK2 signaling in hematopoietic Ba/F3 cells expressing MPL. LnK also inhibits cytokine-independent growth and signaling conferred by JAK2 V617F in those cells. LnK, via its SH2 domain, PH domain, and other regions, associates with JAK2 and JAK2 V617F. Additional LnK domains are involved in LnK downregulation of JAK2 V617F constitutive activation. Elucidating the pathways that attenuate JAK2 and JAK2 V617F signaling provides insight into myeloproliferative disorders and helps to develop therapeutic approaches. Inhibition of Lnk enhances the expression of hematopoetic stem cells and hematopoetic progenitor cells.

### GOVERNMENT RIGHTS

The invention described herein arose in the course of or under NIH Grant No. C/EBP CA026038-29. The United States Government may thus have certain rights in this invention.

### FIELD OF INVENTION

The invention relates to compositions and methods for the treatment of myeloproliferative disorders (MPD) in mammals. Therapeutic approaches include impacting the involvement of LnK in the signaling pathways of mammals having MPD.

### BACKGROUND OF THE INVENTION

Cytokines regulate the proliferation and differentiation of hematopoietic cells by binding to cell surface cytokine receptors. The homodimeric type I cytokine receptors lack intrinsic catalytic activity but mediate ligand-dependent protein phosphorylation through association with tyrosine kinases of the Janus kinase (JAK) family. Of the four family members, JAK2 is prominent both in normal hematopoiesis and in hematological malignancies (Khwaja, 2006; Valentino et al., 2006). The JAK2 mutation, JAK2 V617F, is a somatic mutation identified at high frequency in MPD (Baxter et al., 2005; James et al., 2005; Levine et al., 2005; Kralovics et al., 2005; Zhao et al., 2005). It is present in almost all patients with polycythemia vera (PV), and in approximately half of those with essential thrombocytosis (ET) and idiopathic myelofibrosis (IMF). JAK2 V617F has increased tyrosine kinase activity, and is able to activate JAK-STAT signaling and transform hematopoietic cells, providing it is co-expressed with homodimeric type I cytokine receptors, EPOR, MPL (TPOR) or GCSFR (James et al. 2005; Levine et al., 2005; Zhao et al., 2005; Lu et al., 2005). Furthermore, in murine models, retroviral expression of JAK2 V617F recapitulates the features of PV (James et al., 2005; Lacout et al., 2006; Wernig et al., 2006).

Like other cytokine induced signaling, JAK2 activation is tightly controlled. One mechanism used by cells to regulate the magnitude and duration of JAK2 stimulation is through adaptor proteins that bind JAK2 and its cognate receptor (Khwaja, 2006; Valentino et al., 2006). The adaptor protein LnK is highly expressed in hematopoietic cells and mediates key signaling pathways downstream of several cytokine receptors in these cells (Takaki et al., 2000; Nobuhisa et al., 2003; Takaki et al., 2003). Studies with knockout mice demonstrated that LnK inhibits c-KIT in immature B cells, MPL in megakaryocytes and EPOR, as well as EPOR stimulation of JAK2 in erythroblasts (Takaki et al., 2002; Velazquez et al., 2002; Tong et al., 2004; Tony et al., 2005). In addition, LnK is a negative regulator of self renewal in hematopoietic stem cells (HSC) (Ema et al., 2005; Buza-Vidas et al., 2006; Takizawa et al., 2006; Seita et al., 2007). LnK together with SH2-B and APS form a family of proteins that share a common domain structure including a dimerization domain, a pleckstrin homology (PH) region and a Src homology 2 domain (SH2) (Huang et al., 1995; Takaki et al., 1997; Li et al., 2000). The latter binds phosphotyrosines in various signal-transducing proteins and is critical for LnK inhibition of c-KIT, MPL and EPOR signaling (Nobuhisa et al., 2003; Tong et al., 2004; Tony et al., 2005).

SH2-B and APS are well recognized JAK2 regulators in various signaling networks (O'Brien et al., 2002; Dhe-Pagnon et al., 2004; Maures et al., 2007; Ren et al., 2007). While LnK inhibits MPL and EPOR, both of which depend on JAK2 for signaling, a direct role for LnK in regulating JAK2 has not been demonstrated. Accordingly, there is a need in the art to determine whether LnK can modulate the activity of wild type JAK2 (JAK2 WT) and mutant JAK2 V617F associated with MPD, and to develop a composition and method of treatment of mammals suffering from MPD.

### SUMMARY OF THE INVENTION

The present invention relates to compositions and methods useful in the treatment of MPD. Particular embodiments of the present invention relate to the treatment of MPD by impacting the involvement of LnK in the cytokine receptor signaling pathways of mammals having MPD. Additional embodiments of the present invention can be implemented by one of skill in the art based upon the disclosure and examples provided herein.

### BRIEF DESCRIPTION OF THE FIGURES

FIG. 1A illustrates data representing 3 independent experiments and shows that LNK inhibits proliferation of Ba/F3-MPL cells overexpressing JAK2 WT or JAK2 V617F. In the experiments, Ba/F3 cells stably expressing MPL are co-transfected by electroporation with JAK2 WT (JAK2WT) together with either empty vector (EV), wild type LnK (LNKWT) or LnK SH2 mutant (LNKRE). Two days later, cells are cultured in G418 selection with Tpo (1 ng/ml). Proliferation is measured by cell counts. Data represent the mean±SD of duplicate samples.

FIG. 1B illustrates data representing 3 independent experiments and shows that LnK inhibits proliferation of Ba/F3-MPL cells overexpressing JAK2 WT or JAK2 V617F. In the experiments, Ba/F3 cells stably expressing MPL are co-transfected by electroporation with JAK2 V617F (JAK2VF) together with either empty vector (EV), wild type LnK (LNKWT) or LnK SH2 mutant (LNKRE). Two days later, cells are cultured in G418 selection without Tpo. Proliferation is measured by cell counts. Data represent the mean±SD of duplicate samples.

FIG. 2A illustrates that LnK inhibits STAT5 phosphorylation in Ba/F3-MPL cells overexpressing JAK2 WT and JAK2 V617F. Ba/F3 cells stably expressing MPL are transfected with empty vector (EV), JAK2 WT (JAK2WT) or JAK2 V617F (JAK2VF). Two days later, cells are depleted of serum and cytokines for 4 h and then either treated with Tpo (1 ng/ml, 30 min) or left untreated as indicated. Protein lysates are immunoprecipitated with STAT5 antibody and analyzed by Western blot with phospho-STAT5 antibody (upper panels). Total STAT5 levels are detected with STAT5 antibody (bottom panels).

FIG. 2B illustrates that LnK inhibits STAT5 phosphorylation in Ba/F3-MPL cells overexpressing JAK2 WT and JAK2 V617F. Ba/F3 cells stably expressing MPL are co-transfected by electroporation with JAK2 WT (JAK2WT) together with either empty vector (EV), wild type LnK (LNKWT) or LnK SH2 mutant (LNKRE). Two days later, cells are depleted of serum and cytokines for 4 h and then either treated with Tpo (1 ng/ml, 30 min) or left untreated as indicated. Protein lysates are immunoprecipitated with STAT5 antibody and analyzed by Western blot with phospho-STAT5 antibody (upper panels). Total STAT5 levels are detected with STAT5 antibody (bottom panels).

FIG. 2C illustrates that LnK inhibits STAT5 phosphorylation in Ba/F3-MPL cells overexpressing JAK2 WT and JAK2 V617F. Ba/F3 cells stably expressing MPL are co-transfected by electroporation with JAK2 V617F (JAK2VF) together with either empty vector (EV), wild type LnK (LNKWT) or LnK SH2 mutant (LNKRE). Two days later, cells are depleted of serum and cytokines for 4 h and then either treated with Tpo (1 ng/ml, 30 min) or left untreated as indicated. Protein lysates are immunoprecipitated with STAT5 antibody and analyzed by Western blot with phospho-STAT5 antibody (upper panels). Total STAT5 levels are detected with STAT5 antibody (bottom panels).

FIG. 3A illustrates that LnK inhibits phosphorylation of JAK2 WT and JAK2 V617F. 293T cells are transfected with combinations of empty vector (EV), MPL, JAK2 WT (JAK2WT), JAK2 V617F (JAK2VF), wild type LnK (LNKWT) or SH2 mutant LnK (LNKRE) as indicated. Two days later cells are untreated (panel A) and protein lysates are analyzed by Western blots with phospho-JAK2 antibody (upper panels) and then JAK2 antibody (bottom panels).

FIG. 3B illustrates that LnK inhibits phosphorylation of JAK2 WT and JAK2 V617F. 293T cells are transfected with combinations of empty vector (EV), MPL, JAK2 WT (JAK2WT), JAK2 V617F (JAK2VF), wild type LnK (LNKWT) or SH2 mutant LnK (LNKRE) as indicated. Two days later cells are treated with Tpo (1 ng/ml, 15 or 30 min, panel B) and protein lysates are analyzed by Western blots with phospho-JAK2 antibody (upper panels) and then JAK2 antibody (bottom panels).

FIG. 3C illustrates that LnK inhibits phosphorylation of JAK2 WT and JAK2 V617F. 293T cells are transfected with combinations of empty vector (EV), MPL, JAK2 WT (JAK2WT), JAK2 V617F (JAK2VF), wild type LnK (LNKWT) or SH2 mutant LnK (LNKRE) as indicated. Two days later cells are treated with Tpo (1 ng/ml, 15 or 30 min, panel C) and protein lysates are analyzed by Western blots with phospho-JAK2 antibody (upper panels) and then JAK2 antibody (bottom panels).

FIG. 3D illustrates that LnK inhibits phosphorylation of JAK2 WT and JAK2 V617F. 293T cells are transfected with combinations of empty vector (EV), MPL, JAK2 WT (JAK2WT), JAK2 V617F (JAK2VF), wild type LnK (LNKWT) or SH2 mutant LnK (LNKRE) as indicated. Two days later cells are untreated (panel D) and protein lysates are analyzed by Western blots with phospho-JAK2 antibody (upper panels) and then JAK2 antibody (bottom panels).

FIG. 4A illustrates that LnK interacts with JAK2 WT and JAK2 V617F. 293T cells are co-transfected with combinations of JAK2 WT (JAK2WT), JAK2 V617F (JAK2VF), wild type LnK (LNKWT) or SH2 mutant LnK (LNKRE) as indicated. Protein lysates are immunoprecipitated (IP) with antibodies to JAK2 and analyzed by Western blot with LnK antibody (upper panel). JAK2 and LnK levels in the lysates are analyzed by Western blot with JAK2 and LnK antibodies (bottom panel).

FIG. 4B illustrates that LnK interacts with JAK2 WT and JAK2 V617F. 293T cells are co-transfected with combinations of JAK2 WT (JAK2WT), JAK2 V617F (JAK2VF), wild type LnK (LNKWT) or SH2 mutant LnK (LNKRE) as indicated. Protein lysates are immunoprecipitated (IP) with antibodies to phosphotyrosine and analyzed by Western blot with LnK antibody (upper panel). JAK2 and LnK levels in the lysates are analyzed by Western blot with JAK2 and LnK antibodies (bottom panel).

FIG. 4C illustrates that LnK interacts with JAK2 WT and JAK2 V617F. Protein lysates from 293T cells transfected with either JAK2 WT or JAK2 V617F are incubated with either GST protein or GST-LnK SH2 fusion protein (GST-LNKSH2). GST-protein complexes are analyzed by Western blot with JAK2 antibody. Input represents 1/10 of the lysate used for the pull downs.

FIG. 5 illustrates that the LnK PH domain associates with and inhibits JAK2 V617F. PH means Pleckstrin Homology domain; SH2 means Src Homology 2 domain; DD means dimerization domain.

### DETAILED DESCRIPTION OF THE INVENTION

All references cited herein are incorporated by reference in their entirety as though fully set forth.

Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Myeloproliferative disorders” (MPD) include, but are in no way limited to, bone marrow disorders, chronic myelogenous leukemia, myelofibrosis, polycythemia vera, and thrombocytosis. Myeloproliferative disorders are a group of conditions that cause an overproduction of blood cells, including, platelets, white blood cells, and red blood cells in the bone marrow.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Therapeutically effective amount” as used herein refers to that amount which is capable of achieving beneficial results in a mammal with a myeloproliferative disorder. A therapeutically effective amount can be determined on an individual basis and will be based, at least in part, on consideration of the physiological characteristics of the mammal, the type of delivery system or therapeutic technique used and the time of administration relative to the progression of the disease.

“Treatment” and “treating,” as used herein refer to both therapeutic treatment and prophylactic or preventative measures, wherein the object is to prevent, slow down and/or lessen the disease even if the treatment is ultimately unsuccessful.

The invention includes compositions and methods useful in the treatment of MPD. Particular embodiments of the present invention relate to the treatment of MPD by impacting the involvement of LnK in the cytokine receptor signaling pathways of mammals having MPD. While not wishing to be bound by any particular theory, it is believed that the tyrosine kinase Janus kinase 2 (JAK2) associates with cytokine receptors and is essential for signal transduction in various cells including hematopoietic cells. The JAK2 mutation, JAK2 V617F, found at high frequency in MPD confers cytokine-independent proliferation and constitutive activation of downstream signaling pathways, when co-expressed with homodimeric type I cytokine receptors. The adaptor protein LnK is a negative regulator of several hematopoietic cytokine receptors including the homodimeric type I receptors, EPOR and MPL. LnK attenuates wild type JAK2 signaling in hematopoietic Ba/F3 cells stably expressing MPL. LnK also inhibits cytokine-independent growth and signaling conferred by JAK2 V617F in those cells. LnK via its SH2 domain and other regions associates with JAK2 and mutant JAK2 V617F. While the SH2 domain is necessary for LnK mediated inhibition of MPL-JAK2 signaling, additional LnK domains are involved in LnK downregulation of JAK2 V617F constitutive activation. The elucidation of the cellular pathways that attenuate wild type and mutant JAK2 signaling provides insight into the pathogenesis and therapeutic treatment of MPD.

By targeting some of the major cytokine receptor signaling pathways (i.e. c-KIT MPL and EPOR), LnK plays critical nonredundant roles in hematopoietic cells. JAK2 is an additional LnK target. LnK modulates the activity of JAK2 V617F and may therefore, be implicated in the pathogenesis of JAK2 V617F-positive MPD.

LnK inhibits JAK2 activity by direct and indirect mechanisms. LnK family member, SH2-B, is a potent JAK2 activator, and two models are proposed to explain the mechanism of JAK2 regulation by this adaptor protein. The first is through dimerization-facilitated trans-phosphorylation of JAK2, mediated by the dimerization and the SH2 domains of SH2-B. The second is an allosteric mechanism where the SH2 domain alone is sufficient for JAK2 activation. LnK inhibits the phosphorylation of JAK2 and JAK2 V617F when co-expressed with the type I cytokine receptor, MPL. A mutation disrupting the LnK SH2 domain has a dominant-negative affect, presumably by sequestering endogenous LnK. Since the LnK SH2 domain is required for LnK mediated inhibition of MPL, the inability of the LnK mutant to decrease JAK2 phosphorylation could result from its failure to block MPL. Indeed, in the absence of MPL, the same LnK mutant is more effective than wild type LnK in decreasing JAK2 V617F constitutive activation. These results suggest that similar to SH2-B, LnK SH2 domain enhances, while other LnK domains inhibit JAK2 activation. Physiologically, contrary to SH2-B, for LnK, inhibition is likely the more relevant function as it prevails in wild type LnK.

LnK inhibition of JAK2 involves two mechanisms; one is indirect inhibition of the cytokine receptor that employs JAK2, the second is direct suppression of JAK2 kinase activity. Furthermore, while the receptor mediated inhibition requires the LnK SH2 domain, LnK direct inhibition of JAK2 relies on other LnK domains.

LnK inhibits the JAK2 V617F mutant. Although the JAK2 V617F mutation plays a critical role in the pathogenesis of MPD, it is not the sole event. Several lines of evidence suggest that cooperating events may even precede the JAK2 mutation and determine the course of the disease. The finding that the constitutive active JAK2 V617F mutant is still susceptible to negative regulation by LnK, agrees with other studies indicating that JAK2 V617F is a subtle mutation which only changes the basal activation but not other biological properties of JAK2. Exploring the cellular regulation of JAK2 V617F not only enhances the understanding of the molecular pathogenesis of MPD but paves the way for the development of novel targeted therapies.

In PV the JAK2 V617F mutation occurs in HSC or their progeny, and although the mutation provides a proliferative advantage, it does not confer long-term self-renewal in committed progenitors. Homozygosity for JAK2 V617F as a result of mitotic recombination at 9p, (where JAK2 is located) is an important feature in MPD progression. A significant number of patients with PV and IMF are homozygous for the mutation. In contrast, progenitors having homozygous JAK2 mutation are not found in ET patients. LnK is a critical negative regulator of HSC long-term self-renewal. Therefore, LnK inhibition of both JAK2 WT and JAK2 V617F might play a role in the progression of JAK2 V617F-positive MPD and may also contribute to the observed phenotypic pleiotropy of the disease. Furthermore, our finding that the LnK SH2 domain mutant specifically inhibits JAK2 V617F but not JAK2 WT, may have therapeutic value because one of the challenges facing the development of JAK2 inhibitors is to obtain an inhibitor with preferential activity against mutant rather than wild type JAK2.

In summary, LnK inhibits JAK2 activation, and JAK2 V617F does not escape negative regulation by LnK. Moreover, a molecular mechanism in which different LnK domains function to regulate JAK2 and the JAK2 associated receptor is disclosed. LnK, through attenuation of cytokine receptor signaling, is pivotal for normal hematopoiesis.

In various embodiments, the present invention provides pharmaceutical compositions including at least Lnk along with a pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

Examples of Lnk peptide sequences that may be incorporated in the various pharmaceutical compositions of the present invention are described herein as SEQ. ID. NO.: 3 (homo sapiens), SEQ. ID. NO.: 6 (mus musculus), and SEQ. ID. NO.: 13 (rattus norvegicus). In one embodiment, Lnk has at least 70% identity with respect to the amino acid sequences set forth in SEQ. ID. NO.: 3, SEQ. ID. NO.: 6, and/or SEQ. ID. NO.: 13. In another embodiment, Lnk has at least 80%, 90%, 95%, 96%, 97%, 98% or 99% identity, with respect to the amino acid sequences set forth in SEQ. ID. NO.: 3, SEQ. ID. NO.: 6, and/or SEQ. ID. NO.: 13. Examples of nucleotide sequences encoding Lnk are also described herein as SEQ. ID. NO.: 1 (forward strand, homo sapiens), SEQ. ID. NO.: 2 (reverse strand, homo sapiens), SEQ. ID. NO.: 4 (forward strand, mus musculus), SEQ. ID. NO.: 5 (reverse strand, mus musculus), SEQ. ID. NO.: 11 (forward strand, rattus norvegicus), and SEQ. ID. NO.: 12 (reverse strand, rattus norvegicus).

### EXAMPLES

### Example 1

### Materials and Methods

Expression vectors. MPL and LnK cDNAs are cloned into the retroviral MSCV-GFP and the pcDNA3.1 vectors, respectively. The LnK R392E point mutation is generated by PCR and confirmed by sequencing. pcDNA3.1-JAK2 and pcDNA3.1-JAK2 V617F vectors are obtained from Dr. Zhao (University of Oklahoma, Oklahoma City, Okla.).

Cell culture, expression vectors and transfections. To generate stable cell lines, Ba/F3 cells are transduced with retroviral supernatant containing the MSCV-MPLWT-GFP vector, and then sorted by flow cytometry to isolate GFP-positive cells. Stable Ba/F3-MPL cells are electroporated with different combinations of expression vectors. For growth analysis, two days after electroporation cells are washed in RPMI medium 1640 and then selected in G418 (1 mg/ml) either without or with thrombopoietin (Tpo, 1 ng/ml) for 14 days. Number of viable cells are determined by trypan blue exclusion. 293T cells are transfected using Lipofectamine 2000 (Invitrogen).

Western blot, immunoprecipitation and GST pull-down assays. The following antibodies are used for immunoprecipitation and Western blot analysis: anti-LnK (Serotec); anti-phospho-JAK2, anti-JAK2 and anti-phospho-STAT5 (Cell Signaling Technology); anti-STAT5 (Santa Cruz Biotechnology). GST pull-down assays are carried out with equal amounts of GST and GST-LnK SH2 immobilized on glutathionesepharose beads (Amersham Pharmacia).

As described herein, SEQ. ID. NO.: 7 (forward strand, N terminus), SEQ. ID. NO.: 8 (reverse strand, C terminus), SEQ. ID. NO.: 9 (forward strand, N terminus), SEQ. ID. NO.: 10 (reverse strand, N terminus) provide examples of primers that could be used by one of skill in the art to create GST fusion proteins, which in turn, might be utilized for GST pull-down assays.

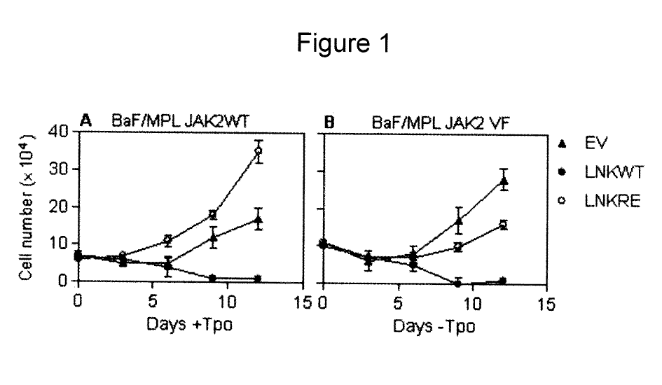

According to the method, LnK inhibits proliferation of Ba/F3-MPL cells expressing either JAK2 WT or JAK2 V617F. Given that transformation by JAK2 V617F requires co-expression with a homodimeric type I cytokine receptor, the effect of LnK on JAK2 signaling in cells expressing one of these receptors is examined. To this end, a murine hematopoietic Ba/F3 cell line, stably expressing the MPL receptor (BaF/MPL) rendering it Tpo responsive can be used. BaF/MPL cells are co-transfected with JAK2 WT and either control empty vector or LnK expression vector, and then cultured under antibiotic selection. Proliferate the control BaF/MPL cells in the presence of Tpo (FIG. 1A). Expression of LnK in these cells significantly attenuates their growth. This is in agreement with earlier studies showing that LnK is a negative regulator of MPL signaling and, that JAK2 WT overexpression cannot overcome LnK-mediated inhibition. The inhibitory functions of LnK have been shown to be mediated mainly through its SH2 domain. A mutation disrupting the LnK SH2 domain, R392E, abolishes the ability of LnK to inhibit growth of BaF/MPL JAK2 WT-transfected cells. In fact, LnK R392E actually increases the proliferation rate of these cells. In a previous study, inactive LnK mutants were shown to have a dominant negative affect by dimerizing with and sequestering endogenously expressed LnK.

To determine whether LnK can block the cytokine independent proliferation of BaF/MPL cells induced by the constitutively active JAK2 V617F, the following can be performed. BaF/MPL cells are co-transfected with JAK2 V617F and LnK and then subjected to antibiotic selection in cytokine-free medium. While expression of JAK2 V617F conferred cytokine-independent growth, cells transfected with JAK2 V617F and LnK do not proliferate, demonstrating that LnK inhibits JAK2 V617F mediated transformation (FIG. 1B). Surprisingly, the SH2 mutant, LnK R392E, only partly compromises the inhibitory effect of LnK, suggesting that regions outside the SH2 domain of LnK are necessary for efficient inhibition.

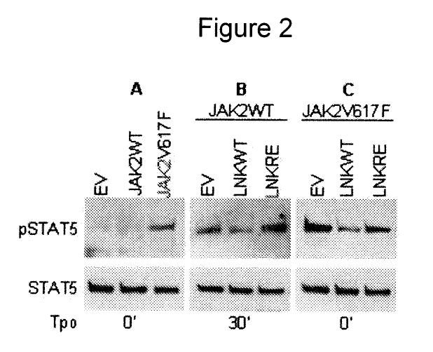

LnK inhibits JAK2 WT and JAK2 V617F signaling in Ba/F3-MPL cells. Binding of Tpo to MPL activates JAK2; the activated JAK2 phosphorylates tyrosines within the receptor cytosolic domain creating docking sites for the binding and subsequent tyrosine phosphorylation of multiple signal-transduction proteins, particularly STATs. To evaluate further the impact of LnK on JAK2 WT and JAK 2V617F activation, STAT5 tyrosine phosphorylation in the BaF/MPL cells is measured. In cells transfected with JAK2 WT, STAT5 activation is not detected in the absence of cytokine stimulation and Tpo treatment induces STAT5 activation (FIG. 2A-B). Expression of LnK, but not LnK R392E, suppresses this induction (FIG. 2B). As expected, expression of JAK2 V617F in BaF/MPL cells results in high levels of cytokine-independent tyrosine phosphorylation of STAT5 (FIG. 2A). Overexpression of LnK and to a lesser extent LnK R392E reduces this phosphorylation (FIG. 2C), showing that LnK down regulates JAK2 V617F constitutive activation and that the SH2 domain, as well as, additional domains of LnK can facilitate this inhibition.

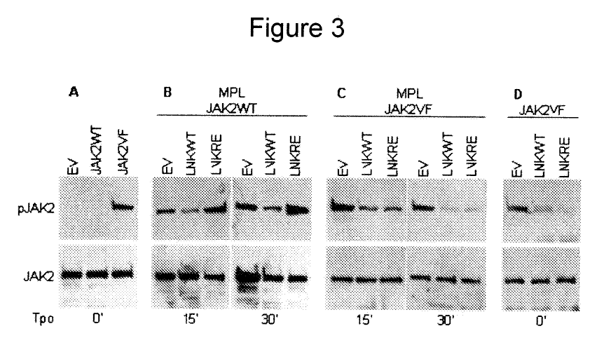

LnK inhibits JAK2 WT and JAK2 V617F activation. For JAK2 to become a fully active tyrosine Y1007 in its kinase domain must be phosphorylated. Determining whether LnK inhibits of JAK2 activation can is examined by measuring the phosphorylation levels of JAK2 tyrosine Y1007/Y1008 in 293T cells transfected with JAK2 and LnK constructs. While JAK2 V617F is constitutively active, autophosphorylation of JAK2 is not detected in these cells (FIG. 3A). To simulate JAK2 activation in 293T cells, similar to the signaling cascade in hematopoietic cells MPL with LnK and either JAK2 WT or JAK2 V617F is co-transfected. Tpo treatment induces JAK2 WT activation and LnK overexpression attenuates this induction (FIG. 3B). On the other hand, LnK SH2 mutant, R392E, increases the levels of tyrosine-phosphorylated JAK2 WT. This result is similar to the findings in the BaF/MPL JAK2 WT expressing cells, where LnK inhibits Tpo-induced proliferation and STAT5 activation, while LnK R392E has the opposite affect. Moreover, consistent with the ability of both LnK and LnK R392E to attenuate JAK2 V617F-induced proliferation and STAT5 phosphorylation in BaF/MPL cells, overexpression of either one of these LnK proteins decreases JAK2 V617F activation in 293T cells (FIG. 3C).

LnK modulates JAK2 activity when JAK2 is co-expressed with MPL. However, MPL itself is a LnK target, raising the possibility that LnK inhibition of JAK2 activity is the consequence of LnK downregulation of MPL. The fact that JAK2 V617F is constitutively active in 293T cells even in the absence of a homodimeric type I cytokine receptor, allows examination of whether LnK can regulate JAK2 activity directly. Overexpression of LnK without co-expression of MPL in 293T cells diminishes JAK2 V617F autophosphorylation, demonstrating that LnK can attenuate JAK2 activity independent of its MPL-mediated inhibition (FIG. 3D). The LnK SH2 domain mutation, R392E, does not compromise LnK ability to inhibit JAK2 V617F activation. In fact, LnK R392E is more potent at decreasing JAK2 V617F phosphorylation than wild type LnK.

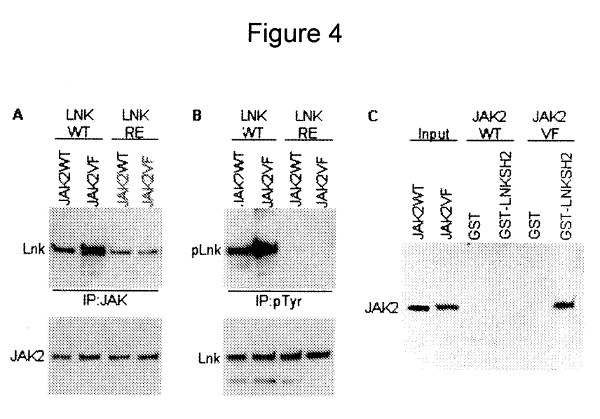

The LnK SH2 domain and other LnK domains associate with JAK2 WT and JAK2 V617F. The above findings suggest that LnK SH2 domain may not only be dispensable but actually impede LnK mediated inhibition of JAK2. Interestingly, LnK family members SH2-B and APS, that share a similar domain structure with LnK, directly bind to phosphorylated tyrosine 813 in JAK2 primarily through their SH2 domains, and these interactions enhance JAK2 activation. However, multiple regions outside the SH2 domains of SH2-B/APS interact at lower affinity with non-phosphorylated JAK2, and these interactions are inhibitory in nature. Co-immunoprecipitate experiments are performed to determine if LnK associates with JAK2. LnK and either JAK2 WT or JAK2 V617F are expressed in 293T cells, and protein lysates are immuprecipitated with a JAK2 antibody. Western blot analysis with LnK antibody shows that LnK is present in both JAK2 WT and JAK2 V617F immunocomplexes (FIG. 4A). The SH2 mutant LnK, R392E, which no longer binds to phosphotyrosine-containing targets, also associates with JAK2 WT and JAK2 V617F, although the interaction is much weaker compared with that between wild type LnK. Notably, the interaction between LnK and JAK2 V617F is stronger compared with that of LnK and JAK2 WT. In contrast LnK, R392E bound to JAK2 WT and JAK2 V617 with similar affinities, indicate that this secondary binding is to non-phosphorylated amino acids in JAK2. GST pull-down assays with a GST-LnK SH2 fusion protein and lysates from 293T cells transfected with either JAK2 WT or JAK V617F show that indeed JAK2 V617F, which is highly phosphorylated in 293T cells, binds to the LnK SH2 domain while JAK2 WT does not (FIG. 4B).

Immuoprecipitations with an anti-phosphotyrosine antibody show that the phosphorylation levels of LnK itself are higher in 293T cells expressing JAK2 V617F compared with cells expressing JAK2 WT (FIG. 4C). As JAK2 V617F kinase activity is greatly increased compared with JAK2 WT, this strongly suggests that LnK, like SH2-B and APS, is a JAK2 substrate. LnK phosphorylation is abolished in the LnK R392E mutant, demonstrating that the LnK SH2 domain is required for its own phosphorylation.

### Example 2

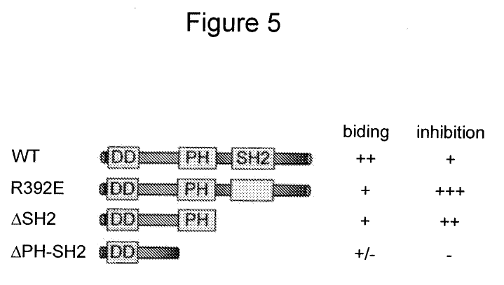

LnK inhibition of JAK2 V671F is assessed. 293T cells are co-transfected with JAK2 V671F and V5-LnK mutant constructs. Interactions between LnK and JAK2 V671F are determined by immunoprecipitation experiments. LnK inhibition of JAK2 V671F is assessed by measuring JAK2 V671F autophosphorylation levels. The LnK PH domain associates with and inhibits JAK2 V671F (FIG. 5).

Expansion of hematopoietic cells has been a long-term therapeutic goal as a way to optimize bone marrow transplantation, as well as gene therapy protocols. The ability to expand hematopoietic stem and progenitor cells (HSC/HPC) ex vivo is limited by their quiescence and is often associated with differentiation and loss of the primitive stem cell phenotype. Ultimately, alternative strategies need to be developed in order to generate sufficient cells for clinical purposes. As shown herein, Lnk is a negative regulator of cytokine receptors which are critical for HSCs/HPCs growth, such as c-Kit and MPL. Significantly, Lnk has not been associated with human disease, and Lnk deficiency in animal models does not result in malignancy or dysfunction of blood cells.

Thus, dominant-negative peptide mimetics of Lnk Pro-rich, pleckstrin homology (PH), or src homology 2 (SH2) domains may be used to inhibit Lnk as an approach to enhance expansion of purified adult or cord-blood HSC/HPC. Additional means to inhibit Lnk include siRNA and small molecules/peptides. Given the compelling evidence for Lnk as a potent regulator of cytokine signaling, Lnk represents a unique target for hematopoietic cell therapeutics without risk of malignant transformation.

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

1. A composition for the treatment of a myeloproliferative disorder in a mammal, comprising:
a therapeutically effective amount of the adaptor protein LnK; and
a pharmaceutically effective carrier.

2. The composition of claim 1, wherein the myeloproliferative disorder is selected from the group consisting of a bone marrow disorder, chronic myelogenous leukemia, myelofibrosis, polycythemia vera and thrombocytosis.

3. The composition of claim 1, wherein the myeloproliferative disorder is selected from the group consisting of idiopathic myelofibrosis, polycythemia vera and essential thrombocytosis.

4. The composition of claim 1, wherein the adaptor protein LnK comprises a polypeptide at least 70% homologous to SEQ ID NO.: 3, SEQ ID NO.: 6 or SEQ ID NO.: 13.

5. The composition of claim 1, wherein the adaptor protein LnK comprises a polypeptide at least 80% homologous to SEQ ID NO.: 3, SEQ ID NO.: 6 or SEQ ID NO.: 13.

6. The composition of claim 1, wherein the adaptor protein LnK comprises a polypeptide at least 90% homologous to SEQ ID NO.: 3, SEQ ID NO.: 6 or SEQ ID NO.: 13.

7. The composition of claim 1, wherein the adaptor protein LnK comprises a polypeptide at least 99% homologous to SEQ ID NO.: 3, SEQ ID NO.: 6 or SEQ ID NO.: 13.

8. The composition of claim 1, wherein the adaptor protein LnK comprises the polypeptide set forth in SEQ ID NO.: 3.

9. The composition of claim 1, wherein the composition is adapted to treat the myeloproliferative disorder through the inhibition of Janus kinase 2.

10. The composition of claim 1, wherein the composition is adapted to treat the myeloproliferative disorder through the inhibition of the Janus kinase 2 mutant JAK2V617F.

11. A composition for the inhibition of a Janus kinase, comprising:
a therapeutically effective amount of the adaptor protein LnK; and
a pharmaceutically effective carrier.

12. The composition of claim 11, wherein the Janus kinase is JAK2.

13. The composition of claim 11, wherein the Janus kinase is the JAK2 mutant JAK2V617F.

14. The composition of claim 11, wherein the adaptor protein LnK comprises a polypeptide at least 70% homologous to SEQ ID NO.: 3, SEQ ID NO.: 6 or SEQ ID NO.: 13.

15. The composition of claim 11, wherein the adaptor protein LnK comprises a polypeptide at least 80% homologous to SEQ ID NO.: 3, SEQ ID NO.: 6 or SEQ ID NO.: 13.

16. The composition of claim 11, wherein the adaptor protein LnK comprises a polypeptide at least 90% homologous to SEQ ID NO.: 3, SEQ ID NO.: 6 or SEQ ID NO.: 13.

17. The composition of claim 11, wherein the adaptor protein LnK comprises a polypeptide at least 99% homologous to SEQ ID NO.: 3, SEQ ID NO.: 6 or SEQ ID NO.: 13.

18. The composition of claim 11, wherein the adaptor protein LnK comprises the polypeptide set forth in SEQ ID NO.: 3.

19. A method of treating a myeloproliferative disorder in mammals, comprising:
administering a composition comprising:
a therapeutically effective amount of the adaptor protein LnK; and
a pharmaceutically acceptable carrier.

20. The method of claim 19, wherein the myeloproliferative disorder is selected from the group consisting of a bone marrow disorder, chronic myelogenous leukemia, myelofibrosis, polycythemia vera and thrombocytosis.

21. The method of claim 19, wherein the myeloproliferative disorder is selected from the group consisting of idiopathic myelofibrosis, polycythemia vera and essential thrombocytosis.

22. The method of claim 19, wherein the composition is adapted to treat the myeloproliferative disorder through the inhibition of Janus kinase 2.

23. The method of claim 19, wherein the composition is adapted to treat the myeloproliferative disorder through the inhibition of the Janus kinase 2 mutant JAK2V617F.

24. The method of claim 19, wherein the adaptor protein LnK comprises a polypeptide at least 70% homologous to SEQ ID NO.: 3, SEQ ID NO.: 6 or SEQ ID NO.: 13.

25. The method of claim 19, wherein the adaptor protein LnK comprises a polypeptide at least 80% homologous to SEQ ID NO.: 3, SEQ ID NO.: 6 or SEQ ID NO.: 13.

26. The method of claim 19, wherein the adaptor protein LnK comprises a polypeptide at least 90% homologous to SEQ ID NO.: 3, SEQ ID NO.: 6 or SEQ ID NO.: 13.

27. The method of claim 19, wherein the adaptor protein LnK comprises a polypeptide at least 99% homologous to SEQ ID NO.: 3, SEQ ID NO.: 6 or SEQ ID NO.: 13.

28. The method of claim 19, wherein the adaptor protein LnK comprises the polypeptide set forth in SEQ ID NO.: 3.

29. A method of enhancing the ex-vivo growth of hematopoetic cells comprising:
inhibiting the cytokine receptor binding of the adaptor protein LnK by administering a dominant negative peptide mimetic of an LnK domain.

30. The method of claim 29, wherein the hematopoetic cells are hematopoetic stem cells.

31. The method of claim 29, wherein the hematopoetic cells are hematopoetic progenitor cells.

32. The method of claim 29, wherein the LnK domain is selected from the group consisting of the Pro-rich domain, the pleckstrin homology domain and the src homology 2 domain.

33. The method of claim 29, wherein the adaptor protein LnK comprises a polypeptide at least 70% homologous to SEQ ID NO.: 3, SEQ ID NO.: 6 or SEQ ID NO.: 13.

34. The method of claim 29, wherein the adaptor protein LnK comprises a polypeptide at least 80% homologous to SEQ ID NO.: 3, SEQ ID NO.: 6 or SEQ ID NO.: 13.

35. The method of claim 29, wherein the adaptor protein LnK comprises a polypeptide at least 90% homologous to SEQ ID NO.: 3, SEQ ID NO.: 6 or SEQ ID NO.: 13.

36. The method of claim 29, wherein the adaptor protein LnK comprises a polypeptide at least 99% homologous to SEQ ID NO.: 3, SEQ ID NO.: 6 or SEQ ID NO.: 13.

37. The method of claim 29, wherein the adaptor protein LnK comprises the polypeptide set forth in SEQ ID NO.: 3.
